(19) United States
(12) Patent Application Publication
(10) Pub. No.: US 2023/0405676 A1
(43) Pub. Date: Dec. 21, 2023

(54) METHODS FOR THE PRODUCTION OF FINE METAL POWDERS

(71) Applicant: US Metals Refining Group, Inc., Centennial, CO (US)

(72) Inventor: Henry W. Kasaini, Centennial, CO (US)

(21) Appl. No.: 18/253,645

(22) PCT Filed: Nov. 23, 2021
(86) PCT No.: PCT/US2021/060616

§ 371 Date: May 19, 2023

Related U.S. Application Data
(60) Provisional application No. 63/116,986, filed on Nov. 23, 2020.

(51) Int. Cl. B22F 9/30; B22F 9/22; B22F 1/05; B22F 1/142; C07C 67/00

(52) U.S. Cl. CPC B22F 9/30; B22F 9/22; B22F 1/05; B22F 1/142; C07C 67/00; B22F 2998/10; B22F 2304/10

(57) ABSTRACT

Methods for the manufacture of fine metal powders from metal-containing ammonium compounds such as ammonium oxalate metal salts. The method includes decomposing particulates of the ammonium oxalate metal salt by heating to a decomposition temperature in the presence of a dilute hydrogen gas to decompose the ammonium oxalate compound, and form a fine metal powder by heating to a higher refining temperature to remove contaminants from the fine metal powder. The method may include the conversion of a non-oxalate metal compound to a hydrated metal oxalate and the dehydration of the hydrated metal oxalate before decomposition to the metal. The method is applicable to the production of a wide variety of metals of high purity and fine particle size.

### CROSS-REFERENCE TO RELATED APPLICATIONS

This application claims the priority benefit of U.S. Provisional Application No. 63/116,986 by Kasaini and filed on Nov. 23, 2020, which is incorporated herein by reference in its entirety.

### FIELD

This disclosure relates to the field of powder metallurgy, and in particular relates to the production of metal powders from metal compounds, and to the production of the precursor metal compounds.

### BACKGROUND

Metal powders are utilized to fabricate a wide variety of products. For many products, the metal powders must have a fine particle size (e.g., of 100 μm diameter or lower) and must have a high purity, e.g., a low concentration of contaminants. In most cases, elemental metals do not occur naturally in large quantities, and the metal powders must be produced from compounds containing the metals such as metal oxides, metal carbonates, and the like.

However, most industrial processes for reducing metal compounds to the metal require high capital costs and/or operating costs. For example, one method for the reduction of rare earth compounds (e.g., rare earth oxides, rare earth chlorides) to rare earth metals involves dispersing the rare earth compound in a molten salt bath (e.g., a molten chloride salt bath) containing a reducing metal, and separating the rare earth metal from the bath after the rare earth compound has been reduced. Other commercialized methods involve the leaching (e.g., dissolution) of the rare earth compound in an inorganic acid followed by solvent extraction to purify the metal salt solution. The purified metal salt solution is then subjected to electrolysis to form a bulk metal, which is then melted and atomized to form a metal powder.

US Patent Publication No. 2020/0047256 by Kasaini discloses methods for the production of fine metal powders from metal carboxylate compounds, such as from metal oxalate single salts, i.e., oxalate salts consisting of oxalic acid and a metal. It is disclosed that fine metal particulates of rare earth metals (e.g., La, Nd, Pr, etc.) and base metals (e.g., Fe, Co, Ni, etc.) may be produced by reduction of the metal oxalate single salts to the metals, e.g., by reduction in nitrogen and/or hydrogen gas.

### SUMMARY

It has been found that several metals may be difficult to form from the metal oxalate single salts disclosed in US Patent Publication No. 2020/0047256 by Kasaini. Broadly characterized, it has been found that metal oxalate single salts with a relatively high concentration of oxygen moieties are difficult to reduce to the metal. That is, a metal that forms multiple bonds with both oxalate (C₂O₄⁻) and oxygen (O²⁻) anions in its valance orbital has a higher energy barrier to decomposition than a metal which is bonded solely to oxalate anions. Examples of such metals include, but are not limited to, niobium (Nb), titanium (Ti), vanadium (V), aluminum (Al), zirconium (Zr), hafnium (Hf), and tantalum (Ta).

By way of example, a single salt of niobium oxalate (C₁₀H₅NbO₂₀) has an excess concentration of bonded oxygen atoms compared to the double salt of ammonium niobate oxalate (C₄H₄NNbO₉). In another example, there is a higher concentration of oxygen moieties in the titanium oxalate single salt (Ti(OH)₂(C₂O₄)₂) than there are in the ammonium titanyl oxalate double salt (NR₄)₂TiO(C₂O₄)₂. It has been found that more expedient to convert the ammonium niobate oxalate or ammonium titanyl oxalate salts to metal powder under conditions of pressure, temperature and feed rate in an inert/hydrogen atmosphere. Additionally, mixed oxalate double salts of more than two metals are amenable to conversion into pure metal alloy powders such as Ti-6% Al-4% V(Ti64), Ti-6% Al-7% Nb and AlNiCo powders.

One embodiment of the present disclosure is directed to the production of oxalate double salts, particularly ammonium oxalate salts. Another embodiment of the present disclosure is directed to a method for the production of fine metal powders from ammonium oxalate metal salts, e.g., from double oxalate salts of ammonium and a metal. Another embodiment of the present disclosure is directed to the refinement (e.g., purification or polishing) of a fine metal powder through the introduction of a reducing compound such as hexamethylenetetramine.

### DESCRIPTION OF THE DRAWINGS

FIG. 1 illustrates a schematic flowsheet of a method for the production of ammonium oxalate metal salts from metal oxides according to an embodiment of the present disclosure.

FIG. 2 illustrates a schematic flowsheet of a method for the production of a fine metal powder from a metal oxalate double salt according to an embodiment of the present disclosure.

FIGS. 3A and 3B illustrate SEM photomicrographs of fine titanium metal powder produced according to the present disclosure.

FIGS. 4A and 4B illustrate the particle size distributions for two fine metal powders produced according to the present disclosure.

### DESCRIPTION OF THE EMBODIMENTS

In a first embodiment, a method for the production of ammonium oxalate metal salts is disclosed. The method is useful for the production of such metal salts from a metal oxide powder, for example. In one embodiment, the method includes contacting a metal oxide powder with an acid to form an acidic solution comprising the solubilized metal and contacting the acidic solution with oxalic acid to precipitate a metal oxalate compound to form an acidic slurry of the metal oxalate compound (e.g., a suspension). A portion of the water is removed from the slurry to concentrate the metal oxalate compound. The concentrated slurry is contacted with ammonium oxalate to form an ammonium oxalate metal salt (e.g., a double salt). The ammonium oxalate metal salt may be separated and dried to form dried ammonium oxalate metal salt particulates, e.g., a dry powder.

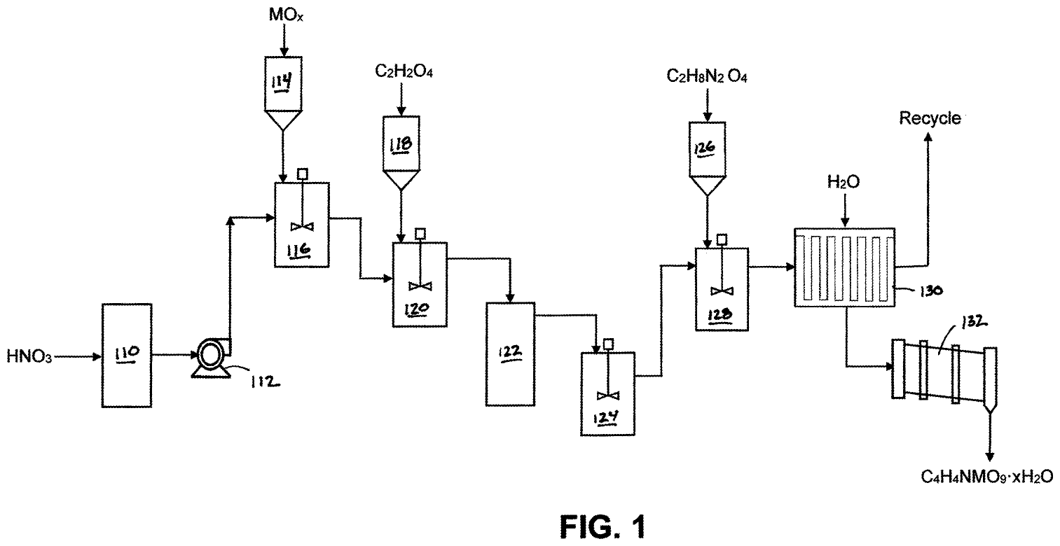

FIG. 1 schematically illustrates a flowsheet for the production of metal ammonium oxalate precipitates (e.g., metal salts) in accordance with one implementation of this embodiment. Acid is supplied to a storage tank 110. As illustrated in FIG. 1, the acid is nitric acid (HNO₃). However, other acids (e.g., mineral acids) may be used including, but not limited to, hydrochloric acid (HCl), sulfuric acid (H₂SO₄), and combinations of such acids. The selected acid should be capable of solubilizing (e.g., dissolving) the metal oxide (generically referred to herein as “MOX”) that is used as the source for the metal. For example, Nb₂O₅ is soluble in nitric acid, but is not highly soluble in hydrochloric acid. The acid should also be of very high purity (e.g., at least about 99.9% purity) to avoid the introduction of contaminants into the system. The acid may have a strength (e.g., an acidity) of at least about 0.5M, such as at least about 1.0M. Generally, the strength of the acid does not have to exceed about 5.0M to sufficiently dissolve the metal oxide, however the selected acidic strength will depend upon several factors including the type of acid selected and the metal oxide(s) to be dissolved.

The acid is transferred from the storage tank 110 to a leaching tank 116, e.g., by a pump 112, as needed. The metal oxide is stored in a hopper 114 (e.g. as a metal oxide powder) until being transferred to the leaching tank 116 as needed, where it is contacted with the acid. The metal oxide may be selected, for example, from base metal oxides including, but not limited to, Nb₂O₅, TiO₂, V₂O₅, Al₂O₃, ZrO₂, HfO, Ta₂O₅ and combinations thereof. It is desirable that the metal oxide(s) have a high purity to reduce the likelihood of contaminants being introduced into the system. In one characterization, the metal oxide has a purity of at least about 98%, such as at least about 99%, such as at least about 99.9% or even at least about 99.99%. The metal oxide and the acid are maintained in contact for a period of time sufficient to dissolve (e.g., solubilize) the metal oxide into the acid to form an acidic solution comprising the metal ions. In one characterization, at least about 99 wt. % of the metal oxide is dissolved in the acid, such as at least about 99.9 wt. %. The leaching tank 116 may be heated if desired to facilitate the dissolution of the metal oxide.

Once the metal oxide has been sufficiently dissolved in the acid, the resulting solution is transferred from the leaching tank 116 to a mixing tank 120 where it is contacted (e.g., mixed) with oxalic acid (C₂H₂O₄). In this regard, oxalic acid may be stored (e.g. as a crystalline powder) in hopper 118 and may be transferred to the mixing tank 120 as needed. It is generally desired to provide an excess (e.g., a stoichiometric excess) of oxalic acid to the mixing tank 120 relative to the solubilized metal ions. In one characterization, the ratio of oxalate (C₂O₄²⁻) to metal ion in the mixing tank 120 is maintained at a level of at least about 1.5:1, such as at least about 1.8:1. In one characterization, the ratio is about 2:1. Generally, the ratio of oxalate to metal ions should not exceed about 3:1. The mixing step (e.g., contacting step) in the mixing tank 120 may be carried out at about atmospheric pressure (e.g. about 1 bar), or may be carried out at an elevated pressure (e.g. above ambient pressure), such as up to about 2 bar for example. The components are mixed for a period of time to form a homogenous acidic solution of the oxalic acid (e.g., the oxalate ion) and the solubilized metal ions.

The acidic solution may then be transferred to an evaporator 122 where the acidic solution is heated to remove water and concentrate the oxalic acid without precipitating appreciable amounts of the metal ions, e.g., without precipitating metal oxalate particulates. For example, the evaporator 122 may be maintained at a temperature of about, or slightly below, the boiling point of water, e.g., at or slightly below 100° C. Some free acid (e.g., HCl or HNO₃) may also evaporate from the solution. In one characterization, the evaporation step reduces the volume of the acidic solution by at least about 25%, such as by at least about 45%. Because the volume is reduced without precipitating metal oxalates, the subsequent treatment vessels may be reduced in volume, reducing capital costs and operating costs for the downstream process operations.

From the evaporator 122, the now concentrated acidic solution of oxalate ions and metal ions is optionally stored in another mixing tank 124 before being transferred to a precipitation tank 128. In the precipitation tank 128, the acidic solution is contacted with ammonium oxalate (e.g., crystalline ammonium oxalate particles) from a hopper 126. Upon contact with the ammonium oxalate, a double salt of metal ammonium oxalate crystals (e.g., (NR₄)₃MO(C₂O₄)₃·H₂O, where M is the metal) will precipitate from the acidic solution. Preferably, the ammonium oxalate from the hopper 126 has a very high purity to reduce the contaminants being introduced into the system. For example, the ammonium oxalate may have a purity of at least about 99%, such as at least about 99.5% or even at least about 99.9%. The precipitation tank 128 may be maintained at a temperature of at least about 5° C. and not greater than about 15° C. to facilitate precipitation of the ammonium oxalate metal salt from the acidic solution. In this regard the tank 128 may be chilled during the precipitation step if desired to cool the solution to below ambient temperature. Further, the precipitation tank 128 may be maintained at substantially ambient pressure during the precipitation step or may be maintained at a slightly elevated pressure if desired.

The resulting ammonium oxalate metal salt (e.g., crystals of the salt) are then separated from the remaining solution (e.g., from the slurry) and dried. As illustrated in FIG. 1, the slurry of ammonium oxalate metal salts may be removed from the precipitation tank 128 and passed through a filter 130 to separate the ammonium oxalate metal salts (e.g., ammonium metal oxalate crystals) from the solution. Thereafter, the ammonium oxalate metal salts may be dried in a dryer 132 to remove residual free water from the metal salts, e.g., at a temperature of from about 100° C. to about 180° C., e.g., about 150° C.

The dried ammonium oxalate metal salts may be characterized as being highly crystalline. For example, the ammonium oxalate metal salts recovered from the drying step may have a crystallinity (e.g., the percentage of metal salt in the crystalline phase) of at least about 90%, such as at least about 95%, at least about 98% or even at least about 99%. The ammonium oxalate metal salts may also be having a high purity, e.g., a very low concentration of impurities. In one characterization, the ammonium oxalate metal salts have a purity of at least about 98%, such as at least about 99%, such as at least about 99.5%, or even at least about 99.9%. The ammonium oxalate metal salts recovered from the drying step may also be characterized as being in particulate form, e.g., in the form of a powder, e.g., a free-flowing powder.

In another embodiment, the present disclosure is directed to methods for the production of fine metal powders from oxalate metal salts, particularly from ammonium oxalate metal salts, e.g. from a precursor powder comprising the ammonium oxalate metal salts. The method is applicable to the production of a wide range of fine metal powders and is particularly useful for the production of fine powders of metals such as niobium, titanium, vanadium and aluminum. The method is also useful for the production of mixed metal powders, which may be readily fabricated into metal alloys. Examples include, but are not limited to, Ti-6% Al-4% V (Ti64), Ti-6% Al-7% Nb and AlNiCo alloy powders.

In one implementation of this embodiment, the method includes heating particulates of an ammonium oxalate metal salt (e.g., a powder) under a decomposition gas (e.g., in a decomposition gas atmosphere). The decomposition gas may be substantially free of water vapor and oxygen to facilitate decomposition of the metal salt particulates to the corresponding metal. The decomposition gas may include hydrogen (H₂) as a reductant. Nitrogen (N₂) and/or ammonia (NH₄) may be used to dilute the hydrogen and/or facilitate removal of oxygen from the system. The fine metal powder may be of very fine particle size and of high purity. The method is rapid and economical as compared to known methods for the production of fine metal powders of high purity from metal compounds such as metal salts.

The ammonium oxalate metal salt particulates that are reduced (e.g., decomposed) to the fine metal powder comprise an anhydrous ammonium oxalate metal salt, i.e., the ammonium oxalate metal salt comprises little to no water of hydration (i.e., water of crystallization). In one implementation, the anhydrous ammonium oxalate metal salt is decomposed by heating the anhydrous ammonium oxalate metal salt particulates to a first temperature (e.g., a decomposition temperature) and holding the anhydrous ammonium oxalate metal salt particulates under a gas composition (e.g., a decomposition gas), where the decomposition gas and the decomposition temperature are sufficient to decompose the anhydrous ammonium oxalate metal salt to form intermediate metal product particulates and a gaseous ammonium oxalate by-product. While the particulate anhydrous ammonium oxalate metal salt is being heated and is decomposing, the released gaseous ammonium oxalate by-product may be separated from the intermediate metal product particulates. The intermediate particulate metal product, which predominately includes metal particulates, is then heated under a second refining gas composition (e.g., a refining gas composition) to a second temperature (e.g., a refining temperature) that is greater than the decomposition temperature to reduce the concentration of contaminants and form the fine metal powder having a high purity.

The foregoing method results in the formation of a fine metal powder derived from ammonium oxalate metal salt particulates. The ammonium oxalate metal salt may be an anhydrous ammonium oxalate metal salt, i.e., a ammonium oxalate metal salt that includes substantially no water of hydration (i.e., water of crystallization). In this case, the anhydrous ammonium oxalate metal salt particulates can be directly heated under a decomposition gas to decompose the ammonium oxalate metal salt and form the gaseous ammonium oxalate by-product.

In most cases, however, ammonium oxalate metal salts are hydrated, i.e., ammonium oxalate metal salts that include water of hydration. Examples include, but are not limited to, ammonium oxalate compounds of the form (NR₄)₂MeO(C₂O₄)₂·nH₂O, (NR₄)₂MeOC₂O₄·nH₂O, and NH₄MeO(C₂O₄)₂·nH₂O, where n can be 1 to 2, for example, such as 1 or 2. Table I illustrates the concentrations in weight percent for typical hydrated and anhydrous ammonium oxalate metal salts.

TABLE I

| | Hydrated | | | Anhydrous | |
|---|---|---|---|---|---|
| | Ammonium Oxalate | | | Ammonium Oxalate | |
| | Meₓ | (NH₄)ₙ(C₂O₄)_{y} | nH₂O | Meₓ | (NH₄)ₙ(C₂O₄)_{y} |
| Ammonium Oxalate | (wt. %) | (wt. %) | (wt. %) | (wt. %) | (wt. %) |
| (NH₄)₃NbO(C₂O₄)₃•H₂O | 20.87 | 75.08 | 4.05 | 21.75 | 78.25 |
| (NH₄)₂TiO(C₂O₄)₂•H₂O | 16.28 | 77.59 | 6.13 | 17.34 | 82.66 |
| (NH₄)₂VOC₂O₄•H₂O | 24.37 | 67.01 | 8.62 | 26.66 | 73.34 |
| NH₄AlO(C₂O₄)₂•H₂O | 10.58 | 82.36 | 7.06 | 11.38 | 88.62 |

As can be seen in Table I, hydrated ammonium oxalate metal salts typically include from about 5 wt. % to about 15 wt. % water of hydration. According to the present disclosure, it is desirable to dehydrate the hydrated ammonium oxalate metal salt particulates, i.e., to remove the water of hydration and form the anhydrous ammonium oxalate metal salt particulates and water vapor, before decomposition of the ammonium oxalate metal salt particulates. In one embodiment, hydrated ammonium oxalate metal salt particulates are dehydrated by heating the particulates to an elevated dehydration temperature, such as to a temperature of at least about 150° C., such as at least about 180° C., such as at least about 200° C., such as at least about 220° C., such as at least about 240° C., or even at least about 260° C., such as at least about 280° C. Such dehydration temperatures are typically sufficient remove the water of hydration and reduce the size of the ammonium oxalate metal salt particulates as a result of the water loss. The temperature at which water of hydration can be removed from an ammonium oxalate metal salt is also influenced by the pressure under which the heating occurs. In any event, the hydrated ammonium oxalate metal salt particulates should not be subjected to conditions of excess heat and pressure during dehydration that would lead to substantial decomposition of the metal oxalate (e.g., decomposition to the metal) before substantially all of the water of hydration has been removed from the particulates. For most ammonium oxalate metal salts, the temperature during the heating step to remove the water of hydration should not be greater than about 440° C., such as not greater than about 400° C., such as not greater than about 360° C., such as not greater than about 340° C., such as not greater than about 320° C., such as not greater than about 300° C. As with the minimum temperatures for dehydration of the hydrated ammonium oxalate metal salt particulates described above, the maximum desirable temperature for dehydration will be influenced by the pressure under which the heating step is carried out (e.g., the dehydration pressure). In one characterization, the step of heating the hydrated ammonium oxalate metal salt particulates is carried out at a dehydration temperature in the range of from about 240° C. to about 280° C., at about ambient pressure.

It is also desirable to separate the water vapor released from the hydrated ammonium oxalate metal salt particulates during the dehydrating step to prevent the water vapor from recombining with the ammonium oxalate metal salt. For example, a dehydration gas (e.g., a sweep gas) may be moved past (e.g., through) the ammonium oxalate metal salt particulates to separate the released water vapor from the particulates and carry the water vapor out of the reactor. The dehydration gas may comprise an inert gas, e.g., nitrogen, argon, helium etc., and in one characterization the dehydration gas comprises nitrogen, and may consist essentially of nitrogen. The dehydration gas may also comprise relatively small concentrations of hydrogen, such as not greater than about 18% hydrogen, and in one embodiment includes up to about 12% hydrogen, such as up to about 6% hydrogen. It is desirable that the sweep gas have a low oxygen content, and in one implementation the sweep gas comprises not greater than about 1% oxygen, such as not greater than about 0.5% oxygen, such as not greater than about 0.1% oxygen, or even not greater than about 0.05% oxygen. In one characterization, a sealed reactor containing the hydrated ammonium oxalate metal salt particulates is evacuated (e.g., to form a vacuum or near vacuum) and the sweep gas is introduced as the dehydrating step begins (e.g., as the heating begins). The presence of the sweep gas and the generated water vapor may cause the pressure in the reactor to subsequently rise during the dehydration step, e.g., up to about 3 bar.

The step of dehydrating the hydrated ammonium oxalate metal salt particulates should be carried out under an elevated temperature and for a time to remove substantially all of the water of hydration from the hydrated ammonium oxalate metal salt particulates. In one embodiment, the dehydration step removes at least about 95% of the water of hydration from the hydrated ammonium oxalate metal salt particulates, such as at least about 98% of the water of hydration, such as at least about 99% of the water of hydration, at least about 99.5% of the water of hydration, or even at least about 99.9% of the water of hydration from the hydrated ammonium oxalate metal salt particulates.

Once the hydrated ammonium oxalate metal salt particulates have been dehydrated to form anhydrous particulates, or if the metal oxalate particulates are provided in an anhydrous form, the anhydrous ammonium oxalate metal salt particulates are decomposed to form intermediate metal product particulates and a gaseous ammonium oxalate by-product. In one implementation, the decomposition and/or sublimation of the oxalate salts may be carried out by heating the anhydrous ammonium oxalate metal salt particulates to an elevated temperature in the presence of a decomposition gas. In one implementation, the anhydrous ammonium oxalate metal salt particulates are heated to a temperature (e.g., a decomposition temperature) that may be higher than the dehydration temperature. In one embodiment, the decomposition temperature is at least about 320° C., such as at least about 345° C., such as at least about 360° C., such as at least about 400° C., at least about 440° C., at least about 480° C., or even at least about 520° C. The decomposition temperature will typically be not greater than about 720° C., such as not greater than about 700° C., or even not greater than about 680° C. It may be preferable to set several decomposition temperatures in a sequence (e.g., a heating profile with several discrete set points) to allow sufficient time at each set temperature to complete a portion of the decomposition. In one alternative, the heating profile may include heating to a dehydration temperature of about 280° C. and holding for about 12 hours, followed by heating to a decomposition temperature of about 345° C. and holding for about 7 hours.

The decomposition step is typically carried out in the presence of a decomposition gas, e.g., a reducing gas and an inert sweeping gas (e.g., in a H₂/N₂ gas reducing atmosphere) to facilitate the transformative reaction and sublimation of the anhydrous ammonium oxalate metal salt to an intermediate compound or to a metal powder. The decomposition gas should have little to no oxygen, and in one embodiment the decomposition gas comprises not greater than about 0.5% oxygen, such as not greater than about 0.1% oxygen, such as not greater than about 0.05% oxygen, or even not greater than about 0.01% oxygen. The decomposition gas may advantageously comprise hydrogen, e.g., at least about 1% hydrogen, such as at least about 2% hydrogen, such as at least about 3% hydrogen. However, the decomposition gas should be dilute with respect to hydrogen, and in one characterization comprises not greater than about 20% hydrogen, such as not greater than about 18% hydrogen, such a not greater than about 15% hydrogen, or even not greater than about 12% hydrogen. In any case, it is an advantage of the methods disclosed herein that the decomposition gas may be dilute with respect to hydrogen. The required concentration of hydrogen will depend upon several factors, including the metal that is formed.

In addition to hydrogen, the decomposition gas may comprise an inert gas such as nitrogen. In one embodiment, the decomposition gas comprises at least about 50% nitrogen, such as at least about 60% nitrogen, such as at least about 70% nitrogen or even at least about 80% nitrogen. The decomposition gas may also include carbon monoxide (CO), e.g., in addition to hydrogen and nitrogen. In one embodiment, the decomposition gas comprises at least about 1% carbon monoxide, such as at least about 2% carbon monoxide. In another embodiment, the decomposition gas comprises not greater than about 30% carbon monoxide, such as not greater than about 25% carbon monoxide, such as not greater than about 20% carbon monoxide.

In one particular embodiment, the decomposition gas for sublimating and converting the ammonium oxalate metal salt to metal powder comprises nitrogen, hydrogen and carbon monoxide, with not greater than about 0.1% oxygen. In one particular characterization, the decomposition gas comprises from about 4% to about 12% hydrogen, from about 2% to about 20% carbon monoxide, and from about 68% to about 94% nitrogen.

The step of heating the anhydrous ammonium oxalate metal salt particulates to decompose the ammonium oxalate metal salt to the intermediate metal product may be carried out under atmospheric pressure. Improved results, however, may be attained by decomposing the ammonium oxalate metal salt at an elevated pressure (e.g., a decomposition pressure). In one embodiment, the decomposition step may be carried out at an elevated pressure (e.g., above atmospheric) such as at least about 1.5 bar, such as at least about 2 bar, or even at least about 2.5 bar. To avoid unnecessary overpressure, the decomposition pressure may be not greater than about 10 bar, such as not greater than about 8 bar, such as not greater than about 6 bar. In one characterization, the decomposition pressure is at least about 1 bar and is not greater than about 4 bar. As noted above, the gaseous ammonium oxalate by-product (e.g., gaseous (NH₄)₂C₂O₄) is separated from the anhydrous metal oxalate particulates during the decomposition of the ammonium oxalate metal salt. For example, the decomposition gas may be flowed through and/or around the anhydrous ammonium oxalate metal salt particulates.

It is also an advantage that the gaseous ammonium oxalate by-product that is separated from the ammonium oxalate metal salt may be captured for recycle. In one embodiment, the captured gaseous ammonium oxalate by-product is condensed, crystallized, and contacted with a non-oxalate metal compound to form an ammonium oxalate metal salt. For example, the captured ammonium oxalate crystals may be placed into solution and used to convert non-oxalate metal compound particulates to ammonium oxalate metal salt particulates, e.g., through a metathesis reaction. In one characterization, the non-oxalate metal compound comprises a metal chloride compound or a metal sulfate compound. The solution product as a result of this metathesis reaction is either NH₄Cl or (NH₄)₂SO₄, both of which may advantageously be utilized as a fertilizer by-product of the process.

After the decomposition of the anhydrous ammonium oxalate metal salt particulates to form the intermediate metal product, the intermediate metal product may be refined, i.e., may be treated to reduce the concentration of contaminants in the intermediate product, e.g., to reduce the concentration of non-metallic constituents associated with the metal powder. In one embodiment, the intermediate metal product is heated to a temperature (e.g., a refining temperature) that is greater than the decomposition temperature, e.g., greater than the temperature that was used to decompose the ammonium oxalate metal salt. In one embodiment, the intermediate metal product is heated above the decomposition temperature to a refining temperature of at least about 700° C., such as at least about 720° C., such as at least about 750° C., or even at least about 800° C. Heating to excessive temperatures, however, are generally not necessary to provide a substantially contaminant-free fine metal powder. For example, the refining temperature will generally be not greater than about 1250° C., such as not greater than about 1200° C., such as not greater than about 1130° C., or even not greater than about 1000° C.

As with the decomposition step, the refining of the intermediate metal product to form the fine metal powder may be carried out in a reducing atmosphere, e.g., in a refining gas composition. The refining gas composition may have the same composition (e.g., same components and compositional ranges) as is disclosed above for the decomposition gas, and in one embodiment, the refining gas composition is substantially the same as the decomposition gas.

The step of refining the intermediate metal product to remove contaminants and form the fine metal powder may also be carried out at an elevated pressure, e.g., at a refining pressure. For example, the refining pressure may be at least about 2 bar, such as at least about 2.5 bar, or even at least about 3 bar. Typically, the refining pressure will be not greater than about 10 bar, such as not greater than about 8 bar, such as not greater than about 6 bar. Heating under an elevated refining pressure with a dilute hydrogen refining gas composition will facilitate the penetration of the fine metal particles with the refining gas and the removal of contaminants from the metal powder.

The fine metal powder may then be cooled, e.g., passively cooled and/or actively cooled. To avoid the formation of undesirable oxides, the cooling may take place in a reduced oxygen atmosphere, such as an atmosphere comprising not greater than about 1% oxygen, such as not greater than about 0.5% oxygen, such as not greater than about 0.1% oxygen, or even not greater than about 0.05% oxygen. For example, the cooling may take place under an inert gas such as nitrogen. In another methodology, the metal powder is cooled under vacuum conditions, e.g., at a reduced pressure of not greater than about 400 torr.

The fine metal powder produced in accordance with the foregoing method may have a very high purity, e.g., a very low concentration of non-metallic impurities. In one embodiment, the fine metal powder comprises not greater than about 2% non-metallic impurities, such as not greater than about 1% non-metallic impurities, such as not greater than about 0.5% non-metallic impurities, or even not greater than about 0.1% non-metallic impurities. For example, the fine metal powder may comprise very low concentrations of oxygen. In one characterization, the fine metal powder comprises not greater than about 0.2% oxygen, such as not greater than about 0.1 wt. % oxygen, or even not greater than about 0.05% oxygen. In another characterization, the fine metal powder comprises not greater than about 0.2 wt. % carbon, such as not greater than about 0.1% carbon, such as not greater than about 0.08 wt. % carbon, such as not greater than about 0.05 wt. % carbon or even not greater than about 0.01 wt. % carbon.

To facilitate the desired reactions, the steps of heating the anhydrous ammonium oxalate metal salt particulates, of separating the gaseous ammonium oxalate by-product, and/or of heating the intermediate metal product particulates may be carried out while agitating the particulates, e.g., the particulate anhydrous ammonium oxalate and/or the intermediate metal particulates. For example, the process steps may be carried out in a fluidized bed reactor.

A wide variety of fine metal powders may be formed using the foregoing method. In one example, the fine metal powder comprises a metal selected from the group consisting of niobium, titanium, vanadium, aluminum, zirconium, hafnium and tantalum. The method is particularly applicable to the production of a fine metal powder comprising one or more rare earth metals. The method is also useful for the production of a fine metal powder admixture of at least two metals, e.g., by starting the with an admixture of two or more ammonium oxalate metal salts. Such mixed metal powder products are useful for the fabrication (e.g., by sintering) of metal alloy products, including but not limited to magnetic products.

In one characterization, the median (D50) particle size of the fine metal powder may be not greater than about 50 μm, such as not greater than about 30 μm, such as not greater than about 25 μm, such as not greater than about 15 μm, such as not greater than about 10 μm, such as not greater than about 5 μm, or even not greater than about 3 μm. Generally, the median particle size of the fine metal powder will be at least about μm, such as at least about 0.01 μm, such as at least about 0.05 μm. In one characterization, the median particle size of the fine metal powder is at least about 0.01 μm and is not greater than about 10 μm. The fine metal powder may also have a narrow particle size distribution, and the metal powders may have relatively low aspect ratio (i.e., the ratio of the longest dimension to the shortest dimension). The median particle size of the fine metal powder is largely a function of the median particle size of the incoming particulate ammonium oxalate metal salt(s). In this regard, the incoming particulate ammonium oxalate metal salt(s) (e.g., hydrated or anhydrous) may be manipulated to adjust the particle size, such as by separation (e.g., sieving) and/or milling of the metal oxalate particulates before decomposition of the anhydrous ammonium oxalate metal salt particulates. In one embodiment, the median particle size of the anhydrous ammonium oxalate metal salt particulates is not greater than about 400 μm, such as not greater than about 200 μm, such as not greater than about 100 μm, or even not greater than about 50 μm.

In another embodiment, a carbon and nitrogen-rich organic reducing compound is added to facilitate the refinement of metal powders formed from metal compounds, e.g., the refinement of metal powders formed in accordance with the foregoing embodiment, e.g. during the refinement step. The reducing compound is selected to lower the oxygen-content of the final powder and hence improve the purity of the final metal powder. For example, the organic reducing compound may be a methyl complex, such as a methyl nitro oxyl compound. In one characterization, the organic reducing compound comprises hexamethylenetetramine (HMTA, sometimes referred to as methenamine). The organic reducing compound provides excess carbon and nitrogen atoms to react with residual oxygen atoms on the metal powder surface at relatively low temperature, i.e., to “polish” the fine metal powder by scavenging the oxygen.

The organic reducing compound (e.g., HMTA) may be added at any point during the production of the fine metal powder from a metal compound. For example, and referring to the embodiment disclosed above, the organic reducing compound may be added with the hydrated ammonium oxalate metal salt, to the anhydrous ammonium oxalate metal salt, and/or to the intermediate metal powder prior to and/or during the refinement step. In one characterization, the organic reducing compound may be added to the precursor powder (e.g., precursor to the metal) in an amount of at least about 0.1 wt. %, such as at least about 0.5 wt. %, such as at least about 1.0 wt. % or even at least about 2 wt. %. Generally, concentrations of greater than about 10.0 wt. % are not necessary and may be detrimental by leaving excess residual carbon and nitrogen in the fine metal powder. In one particular characterization, the precursor powder includes at least about 3.0 wt. % and not greater than about 6.0 wt. % of the organic reducing compound. It has been found that the organic reducing compound may enable the production of fine metal powders of high purity at not greater than 1100° C., such as not greater than about 1000° C., or even not greater than about 850° C.

In another embodiment, the addition of a small amount of carbon (e.g., graphitic carbon) may also be advantageous for the sequestration of residual oxygen at elevated temperatures during the final conversion of precursor powder to the fine metallic powder. In this regard, up to about 0.5 wt. % free carbon, such as up to about 1.5 wt. % free carbon, or even up to about 2.5 wt. % free carbon may be added to the precursor powder. However, it is preferred that any carbon present in the precursor powder reacts completely during the heating step(s) to form gaseous products, e.g., to form CO and CO₂. A graphite mold (crucible) may also be used to assist in the removal of residual oxygen from the fine metallic powder at elevated temperatures in the furnace.

It will be appreciated that the foregoing embodiments may be implemented individually, or in various combinations. For example, the precursor to the method for forming a fine metal powder (e.g., the anhydrous ammonium oxalate particulates) may be formed using the method disclosed above for the production of ammonium oxalate metal salts. The production of the fine metal powder may include the use of an organic reducing agent and/or carbon, as is disclosed in the embodiments above.

### EXAMPLES

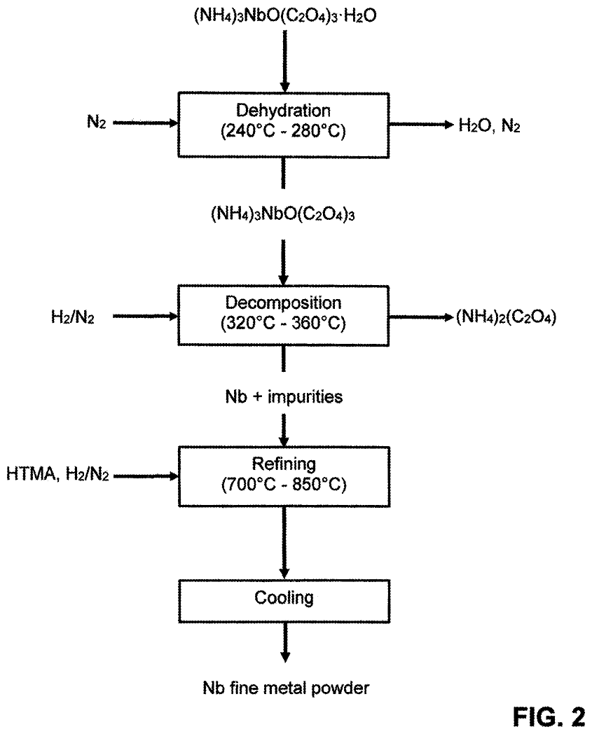

FIG. 2 illustrates a flowsheet for the production of a fine Nb metal powder from niobium ammonium oxalate salt particulates, e.g., from ammonium oxalate salts produced in accordance with the process illustrated in FIG. 1. 100.0000 grams of the metal salt particulates ((NH₄)₃NbO(C₂O₄)₃·H₂O) are first subjected to a dehydration step where the particulates are heated to about 240° C. to about 280° C. and held for about 12 hours under flowing N₂ gas in a fluidized bed reactor. The resulting product is 95.9525 grams of an ammonium niobate oxalate anhydrous metal salt ((NH₄)₃NbO(C₂O₄)₃). The temperature of the reactor containing the anhydrous metal salt is then raised to from about 320° C. to about 360° C. and held for about 7 hours under a flowing decomposition gas of 12% H₂/88% N₂ to remove the oxalic acid from the metal salt.

The resulting product is an Nb metal powder containing impurities, including oxygen. In a subsequent refining step, the temperature of the reactor containing the impure Nb metal powder is increased to a refining temperature of from about 650° C. to about 950° C. to remove the impurities. To facilitate the refining of the metal powder at such temperatures, 0.2 wt. % of HTMA is added to the reactor as an organic reductant compound. Thereafter, the refined (e.g., high purity) Nb metal powder is cooled under an inert cooling gas, resulting in 20.8735 grams of the Nb metal powder. The Nb metal powder has an average particle size of about 10 μm and comprises at least about 99.5% Nb, i.e., has a concentration of contaminants on a trace metal basis of not greater than about 0.5%. Further, the Nb metal powder may be characterized as having an oxygen content of not greater than about 2% and a carbon content of not greater than about 1%.

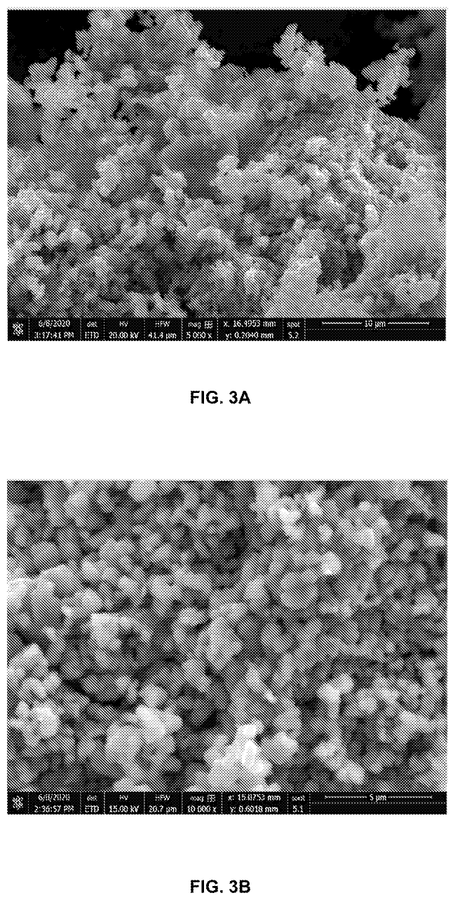

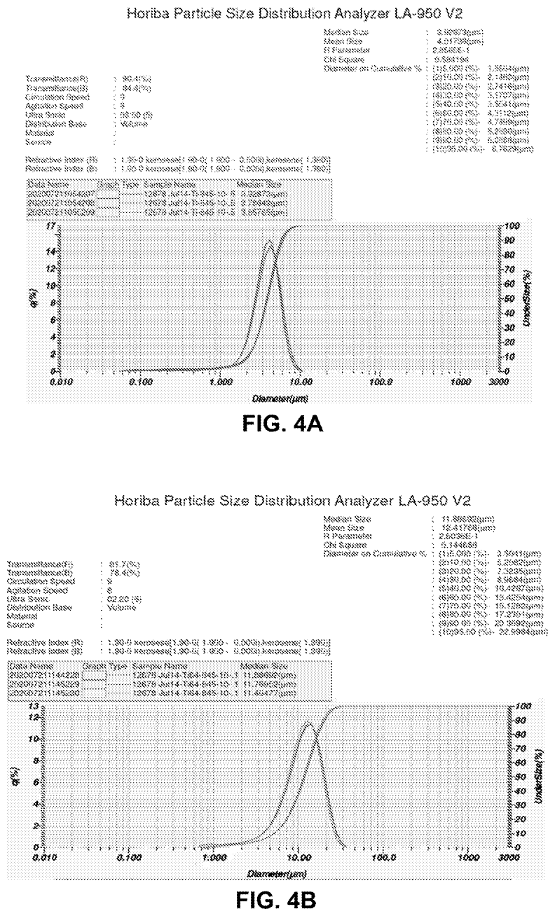

FIGS. 3A and 3B illustrate SEM photomicrographs of fine titanium metal powder produced according to the methods disclosed herein, i.e., by the conversion of particulate (NH₄)₂TiO(C₂O₄)₂·H₂O to fine titanium metal powder. The photomicrographs illustrate that the fine titanium metal powder has a very fine particle size and is highly crystalline. The particle size distribution (PSD) illustrated in FIG. 4A shows that the fine Ti metal powder has a median particle size of about 3.9 μm, and 95 vol. % of the particles have a size of not greater than about 6.8 μm. Such a fine particle size is attained without milling of the powder. FIG. 4B illustrates the PSD for a titanium alloy powder produced according to the present disclosure, namely a Ti-6% Al-4% V alloy (also referred to as “Ti64”). The fine metal powder has a median particle size of about 11.9 μm, and 95 vol. % of the particles have a size of not greater than about 23.0 μm.

While various embodiments have been described in detail, it is apparent that modifications and adaptations of those embodiments will occur to those skilled in the art. However, it is to be expressly understood that such modifications and adaptations are within the spirit and scope of the present disclosure, including the use of known and appropriate engineering vessels and reactors.

What is claimed is:

1. A method for the production of fine metal powder, comprising the steps of:
heating a precursor powder comprising anhydrous ammonium oxalate metal salt particulates to a decomposition temperature while the precursor powder is under a decomposition gas, the decomposition gas and the decomposition temperature being sufficient to decompose the anhydrous ammonium oxalate metal salt particulates and form an intermediate metal powder and a gaseous by-product comprising ammonium oxalate and/or oxalic acid;
separating the gaseous by-product from the intermediate metal powder; and
heating the intermediate metal powder to a refining temperature that is greater than the decomposition temperature and holding the intermediate metal powder at the refining temperature in the presence of a refining gas to remove contaminants in the intermediate metal powder and form a high purity fine metal powder.

2. The method recited in claim 1, further comprising the step of:
before heating to a decomposition temperature, dehydrating hydrated ammonium oxalate metal salts to remove water of hydration from the hydrated ammonium oxalate metal salts and form the anhydrous ammonium oxalate metal salts.

3. The method recited in claim 2, wherein the step of dehydrating the hydrated ammonium oxalate metal salts comprises heating the hydrated ammonium oxalate metal salts to a dehydration temperature.

4. The method recited in claim 3, wherein the dehydration temperature is at least about 240° C.

5. The method recited in any one of claim 3 or 4, wherein the dehydration temperature is not greater than about 340° C.

6. The method recited in any one of claims 2 to 5, further comprising the step of separating water vapor from the hydrated ammonium oxalate metal salts during the dehydrating step.

7. The method recited in claim 6, wherein the step of separating the water vapor from the hydrated ammonium oxalate metal salts comprises moving a dehydration gas through the hydrated ammonium oxalate metal salts.

8. The method recited in claim 7, wherein the dehydration gas comprises nitrogen.

9. The method recited in any one of claim 7 or 8, wherein the dehydration gas comprises not greater than about 0.1% oxygen.

10. The method recited in any one of claims 2 to 9, wherein the step of dehydrating the hydrated ammonium oxalate metal salts removes at least about 99.9% of the water of hydration from the hydrated ammonium oxalate metal salts.

11. The method recited in any one of claims 1 to 10, wherein the decomposition temperature is at least about 360° C.

12. The method recited in any one of claims 1 to 11, wherein the decomposition temperature is not greater than about 700° C.

13. The method recited in any one of claims 1 to 12, wherein the decomposition gas comprises not greater than about 0.01% oxygen.

14. The method recited in any one of claims 1 to 13, wherein the decomposition gas comprises at least about 50% nitrogen.

15. The method recited in any one of claims 1 to 14, wherein the decomposition gas comprises hydrogen.

16. The method recited in claim 15, wherein the decomposition gas comprises not greater than about 18% hydrogen.

17. The method recited in any one of claims 1 to 16, wherein the decomposition gas comprises carbon monoxide.

18. The method recited in claim 17, wherein the decomposition gas comprises at least about 2% carbon monoxide and not greater than about 20% carbon monoxide.

19. The method recited in any one of claims 1 to 18, wherein the decomposition gas comprises nitrogen, hydrogen and carbon monoxide.

20. The method recited in any one of claims 1 to 19, wherein the step of heating the anhydrous ammonium oxalate metal salts is carried out under an elevated decomposition pressure.

21. The method recited in claim 20, wherein the decomposition pressure is at least about 2 bar.

22. The method recited in any one of claim 20 or 21, wherein the decomposition pressure is not greater than about 6 bar.

23. The method recited in any one of claims 1 to 22, wherein the step of separating the gaseous oxalate by-product from the anhydrous ammonium oxalate metal salt comprises moving the decomposition gas through the anhydrous ammonium oxalate metal salts.

24. The method recited in any one of claims 1 to 23, wherein the refining temperature is at least about 720° C.

25. The method recited in any one of claims 1 to 24, wherein the refining temperature is not greater than about 1200° C.

26. The method recited in any one of claims 1 to 25, wherein the refining gas composition comprises not greater than about 0.01% oxygen.

27. The method recited in any one of claims 1 to 26, wherein the refining gas composition comprises at least about 50% nitrogen.

28. The method recited in any one of claims 1 to 27, wherein the refining gas composition comprises hydrogen.

29. The method recited in claim 28, wherein the refining gas composition comprises not greater than about 18% hydrogen.

30. The method recited in any one of claims 1 to 29, wherein the refining gas composition comprises carbon monoxide.

31. The method recited in claim 30, wherein the refining gas composition comprises at least about 2% carbon monoxide and not greater than about 20% carbon monoxide.

32. The method recited in any one of claims 1 to 31, wherein the refining gas composition is substantially the same as the decomposition gas.

33. The method recited in any one of claims 1 to 32, wherein the step of heating the intermediate metal powder is carried out at an elevated refining pressure.

34. The method recited in claim 33, wherein the refining pressure is at least about 2 bar.

35. The method recited in any one of claim 33 or 34, wherein the refining pressure is not greater than about 6 bar.

36. The method recited in any one of claims 1 to 35, wherein the fine metal powder comprises not greater than about 2% non-metallic impurities.

37. The method recited in any one of claims 1 to 35, wherein the fine metal powder comprises not greater than about 1% non-metallic impurities.

38. The method recited in any one of claims 1 to 37, wherein the fine metal powder comprises not greater than about 0.1% oxygen.

39. The method recited in any one of claims 1 to 38, wherein the fine metal powder comprises a metal selected from the group consisting of niobium, titanium, vanadium, aluminum, zirconium, hafnium and tantalum.

40. The method recited in claim 39, wherein the fine metal powder comprises niobium.

41. The method recited in claim 40, wherein the anhydrous ammonium oxalate metal salt comprises niobium ammonium oxalate.

42. The method recited in claim 39, wherein the fine metal powder comprises titanium.

43. The method recited in claim 42, wherein the anhydrous ammonium oxalate metal salt comprises diammonium titanyl oxalate.

44. The method recited in claim 39, wherein the fine metal powder comprises vanadium.

45. The method recited in claim 44, wherein the anhydrous ammonium oxalate metal salt comprises diammonium vanadyl oxalate.

46. The method recited in claim 39, wherein the fine metal powder comprises aluminum.

47. The method recited in claim 46, wherein the anhydrous ammonium oxalate metal salt comprises aluminum ammonium oxalate.

48. The method recited in any one of claims 1 to 47, wherein the fine metal powder comprises at least two metals.

49. The method recited in any one of claims 1 to 48, wherein the fine metal powder has a median (D50) particle size of not greater than about 10 μm.

50. The method recited in claim 49, wherein the fine metal powder has a median (D50) particle size of not greater than about 6 μm.

51. The method recited in claim 50, wherein the fine metal powder has a median (D50) particle size of at least about 1 μm.

52. The method recited in any one of claims 1 to 51, wherein at least the steps of heating the metal-containing anhydrous ammonium oxalate metal salts, separating the gaseous ammonium oxalate by-product and heating the intermediate metal powder are carried out while agitating the anhydrous ammonium oxalate metal salt and the intermediate metal powder.

53. The method recited in claim 52, wherein the agitating is carried out in a fluidized bed reactor.

54. The method recited in any one of claims 1 to 53, wherein the gaseous ammonium oxalate by-product that is separated from the anhydorus ammonium oxalate metal salts is recovered and recycled.

55. The method recited in claim 54, wherein the gaseous ammonium oxalate by-product is condensed and crystallized, and is then contacted with non-oxalate metal salts to form ammonium oxalate metal salts.

56. The method recited in claim 55, wherein the non-oxalate metal salts comprise a metal compound selected from the group consisting of solubilized metal chloride compounds, metal oxide compounds, metal sulfate compounds and metal carbonate compounds.

57. The method recited in any one of claims 1 to 56, comprising the step of cooling the fine metal powder in the substantial absence of oxygen.

58. The method recited in any one of claims 1 to 57, wherein the fine metal powder comprises not greater than about 0.1 wt. % carbon contamination.

59. The method recited in any one of claims 1 to 58, wherein the precursor powder comprises a methyl nitro oxyl compound.

60. The method recited in claim 59, wherein the methyl nitro oxyl compound comprises hexamethylenetetramine (HMTA).

61. The method recited in any one of claim 59 or 60, wherein the precursor powder comprises at least about 0.5 wt. % of the methyl nitro oxyl compound.

62. The method recited in any one of claims 59 to 61, wherein the precursor powder comprises not greater than about 6.0 wt. % of the methyl nitro oxyl compound.

63. A method for the production of an ammonium oxalate metal salt, comprising the steps of:
contacting a metal oxide compound with oxalic acid to form an intermediate oxalate salt;
removing at least a portion of water from the intermediate oxalate salt;
contacting the intermediate oxalate salt with ammonium oxalate to form a slurry;
cooling the slurry to crystallize an ammonium oxalate metal salt; and
separating the ammonium metal oxalate salt from the slurry.

64. The method recited in claim 63, wherein the metal is selected from the group consisting of niobium, titanium, vanadium, aluminum, zirconium, hafnium and tantalum.
